(19) United States
(12) Patent Application Publication
(10) Pub. No.: US 2006/0074050 A1
(43) Pub. Date: Apr. 6, 2006

(54) Composition and method for treating hyperproliferative diseases

(75) Inventors: Yan Chang, Ashland, MA (US); Finbarr Cotter, Horsham (GB)

Correspondence Address: FISH & NEAVE IP GROUP;ROPES & GRAY LLP, ONE INTERNATIONAL PLACE, BOSTON, MA, 02110-2624, US

(73) Assignee: GlycoGenesys, Inc., Boston, MA (US)

(21) Appl. No.: 11/181,201

(22) Filed: Jul. 13, 2005

Related U.S. Application Data
(60) Provisional application No. 60/588,608, filed on Jul. 14, 2004.

(51) Int. Cl. A61K 31/715; A61K 31/704; A61K 31/5513; A61K 31/409; A61K 31/407; A61K 31/165; A61K 31/47; A61K 31/405

(52) U.S. Cl. 514/54; 514/221; 514/310; 514/410; 514/317; 514/621; 514/34; 514/283; 514/419

(57) ABSTRACT

The present invention is directed to methods and compositions for treatment of hyperproliferative diseases. The composition of the invention comprises a carbohydrate having a backbone comprising polygalacturonan and a ligand of peripheral benzodiazepine receptor. The present compositions and methods are used to treat various cancers and other diseases where cells undergo pathological and unwanted proliferation.

### CROSS REFERENCE TO RELATED APPLICATIONS

This application claims the benefit of U.S. Provisional Application No. 60/588,608 filed Jul. 14, 2004, entitled “Composition and Method for Treating Hyperproliferative Diseases,” the disclosure of which is incorporated herein in its entirety.

### BACKGROUND OF THE INVENTION

Conventional treatment for cancers and other diseases involving unwanted cellular proliferation involves the use of chemotherapeutic agents, radiation, and surgery, either alone or in combination. The medical arts have developed a number of treatments based upon the foregoing therapies.

Although chemotherapy has been effective in treating various types of malignancies, many antineoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Treatment of hyperproliferative diseases may be directed to preventing further growth and actively inducing the destruction of the pathological cells. The induction of apoptosis, or programmed cell death, is a common mode of operation for several classes of anticancer drugs.

There is a need for an improvement method for treatment of hyperproliferative diseases to reduce undesirable side effects and enhance the effectiveness of preexisting treatment methods by providing combination of pharmaceutical agents.

### BRIEF SUMMARY OF THE INVENTION

One aspect of the invention provides methods for treating hyperproliferative disease such as cancer by conjointly administering a carbohydrate polymer and a ligand of peripheral benzodiazepine receptors (PBR).

The invention also provides a composition comprising a carbohydrate polymer and a ligand of peripheral benzodiazepine receptors.

An aspect of the invention provides a kit that includes (i) a ligand of peripheral benzodiazepine receptors; (ii) a therapeutically effective amount of a carbohydrate polymer; and (iii) instructions and/or a label.

Another aspect of the invention provides a kit that includes (i) a ligand of peripheral benzodiazepine receptors; (ii) a therapeutically effective amount of a carbohydrate polymer; (iii) a therapeutically effective amount of a chemotherapeutic agent; and (iv) instructions and/or a label.

Still another aspect provides a packaged pharmaceutical including (i) a therapeutically effective amount of a carbohydrate polymer; and (ii) instructions and/or a label for administration of the carbohydrate polymer for the treatment of pateints afflicted with hyperproliferative disease.

The invention also contemplates a method of treatment comprising administering a chemotherapeutic agent in conjunction with the carbohydrate polymer and a ligand of PBR. In a preferred embodiment, the chemotherapeutic agent is a topoisomerase inhibitor. In a more preferred embodiment, the chemotherapeutic agent is etoposide.

A preferred class of carbohydrate to be used in the method of the present invention comprises a carbohydrate with a polymeric backbone, optionally having side chains dependent therefrom. The side chains are terminated by a galactose, rhamnose, xylose, or arabinose unit. This material may be synthetic, natural, or semi-synthetic. In one particular embodiment, the therapeutic compound comprises a partially demethoxylated polygalacturonic acid backbone which may be interrupted with rhamnose residues. In another embodiment, the therapeutic compounds comprise homogalacturonan backbones. Such compounds may be prepared from naturally occurring pectin, and are referred to as partially depolymerized pectin or modified pectin. The most preferred class of carbohydrate for use in the present invention comprises a polygalacturonan backbone with side chains terminating in galactose.

A preferred class of ligands of PBR comprises 1-(2-chlorophenyl)-N-methyl-N-(1-methylpropyl)-3-isoquino-linecarboxamide (PK11195), 7-chloro-5-(4-chlorophenyl)-1,3-dihydro-1-methyl-2-H-1,4-benzodiazepin-2 (4-chlorodiazepam or Ro5-4864), porphyrins, alpidem, N-(2,5-dimethoxybenzyl)-N-5-fluoro-2-phenoxyphenyl)acetamide (DAA1106), 2-aryl-3-indoleacetamide (FGIN-1), diazepam (Valium®), flumazenil (Romazicon®) or lonidamine.

The method of present invention may be administering such materials orally, by injection, transdermally, subcutaneously or by topical application, depending upon the specific type of hyperproliferative disorder being treated, and the adjunct therapy.

### BRIEF DESCRIPTION OF THE FIGURES

FIG. 1 depicts the dose curve of 4-chlorodiazepam to in vitro cell culture with or without concomitant administration of GCS-100.

### DETAILED DESCRIPTION OF THE INVENTION

### I. OVERVIEW

Disclosed herein is a method for enhancing the efficacy of a therapeutic treatment for cancer and other hyperproliferative disorders involving unwanted cellular proliferation, such as psoriasis or rheumatoid arthritis, in a patient by administering a combination of specific carbohydrate materials described herein and an inducer of apoptosis.

Apoptosis is a well-regulated cellular event, involving distinct signaling proteins and defined disruption of cellular structures. A critical event in the process leading to apoptosis is opening of the permeability transition pore (“PT pore”) of mitochondria. The mitochondrial PT pore, or the mega-channel or multi-conductance channel, participates in regulating the level of calcium in the matrix, the pH and the transmembrane potential in the mitochondria. The opening of the PT pore is regulated by Bcl-2, and results in dissipation of the mitochondrial internal transmembrane potential, which leads to disruption of the integrity of the outer membrane and release of mitochondrial intermembrane proteins. The PT pore is regulated by multiple effectors.

It has recently been shown that ligands of peripheral benzodiazepine receptors of the mitochondria also bind to the PT pore and strongly induce apoptosis.

In a preferred embodiment, carbohydrate materials for use in the present invention comprises a polymeric backbone, optionally having side chains dependent therefrom. In a preferred embodiment, the inducer of apoptosis is a ligand of peripheral benzodiazepine receptors (“PBR”) located in the mitochondrial membrane.

In particular embodiments, the methods of invention are carried out by administering a ligand of PBR and a carbohydrate polymers comprising a partially demethoxylated polygalacturonic acid backbone which may be interrupted by rhamnose. The carbohydrate polymer may be homogalacturonan. Alternatively, the carbohydrate polymer may comprise polygalacturonic acid interrupted by rhamnose, having side chains pendent thereof, which are terminated by a galactose, rhamnose, xylose, or arabinose unit.

### II. DEFINITIONS

As used herein, the terms “agent” and “compound” include both protein and non-protein moieties. An agent may be a small organic molecule, a carbohydrate polymer, a polypeptide, a protein, a peptide complex, a peptidomimetic, a non-peptidyl agent, or a polynucleotide.

As used herein, “ameliorates” means alleviate, lessen, or decrease the extent of a symptom or decrease the number of occurrence of episodes of a disease manifestation.

As used herein the term “animal” refers to mammals, preferably mammals such as humans. Likewise, a “patient” or “subject” to be treated by the method of the invention can mean either a human or non-human animal.

The terms “apoptosis” or “programmed cell death,” refers to the physiological process by which unwanted or useless cells are eliminated during development and other normal biological processes. Apoptosis, is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Cells undergoing apoptosis show characteristic morphological and biochemical features. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial dysfunction accompanying apoptosis. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis.”

The “growth state” of a cell refers to the rate of proliferation of the cell and the state of differentiation of the cell.

The term “prevent” or “preventing” is art-recognized, and when used in relation to a condition, such as recurrence or onset of a disease such as cancer and other hyperproliferative diseases or any other medical condition, is well understood in the art, and includes administration of a treatment which reduces the frequency of, or delays the onset of, symptoms of a medical condition in a subject relative to a subject which does not receive the treatment. Thus, prevention of cancer includes, for example, reducing the instances of new primary or metastatic neoplastic growth in a population of patients receiving a prophylactic treatment relative to an untreated control population, and/or delaying the onset of neoplastic growth in a treated population compared to untreated population.

As used herein, “proliferating” and “proliferation” refer to cells undergoing mitosis.

### III. EXEMPLARY EMBODIMENTS

### A. MATERIALS USEFUL TO PRACTICE PRESENT INVENTION

One class of compound contemplated by the present invention is carbohydrate-containing polymers. Materials useful in the present inventions may be generally comprised of natural or synthetic polymers and oligomers. Preferably, such polymers are very low in toxicity.

A preferred class of polymers for the practice of the present invention are carbohydrate-derived polymers, comprising oligomeric or polymeric species of natural or synthetic origin, rich in galactose or arabinose. Such materials will preferably have a molecular weight in the range of up to 500,000 daltons (Da) and, more preferably, in the range of up to 150,000 Da. One particular material comprises a partially demethoxylated polygalacturonic acid backbone which may be interrupted by rhamnose with galactose-terminated side chains pendent therefrom. Another particular material comprises a homogalacturonan backbone with or without side chains pendent therefrom.

One group of materials falling within this general class comprises a partially demethoxylated polygalacturonic acid backbone having rhamnose, galactose, arabinose or other sugar residues pendent therefrom. In certain embodiments, modified pectins useful to practice the invention are described by formulae I and II below, and it is to be understood that yet other variants of this general compound may be prepared and utilized in accord with the principles of the present invention.

1. Homogalacturonan
-[α-D-GalpA-(1→4)-α-D-GalpA]ₙ- (I)

2. Rhamnogalacturonan 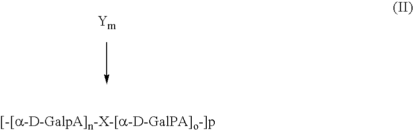
In the formulae above, m is≧0 n, o and p are≧1, X is α-Rhap; and Ym represents a linear or branched chain of sugars (each Y in the chain Ym can independently represent a different sugar within the chain). The sugar Y may be, but is not limited to, any of the following: α-Galp, β-Galp, β-Apif, β-Rhap, α-Rhap, α-Fucp, β-GlcpA, α-GalpA, β-GalpA, β-DhapA, Kdop, β-Acef, α-Araf, β-Araf, and α-Xylp. Ym may be

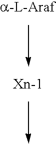

Abbreviated sugar monomer names used herein are defined as follows: GalA: galacturonic acid; Rha: rhamnose; Gal: galactose; GIcA: glucuronic acid; DhaA: 3-deoxy-D-lyxo-heptulosaric acid; Kdo: 3-deoxy-D-manno-2-octulosonic acid; Ace: aceric acid (3-C-carboxy-5-deoxy-L-lyxose); Ara: arabinose. Italicized p indicates the pyranose form, and italicized f indicates a furanose ring.

Another class of compound useful for the present invention are represented by formulae III and IV. 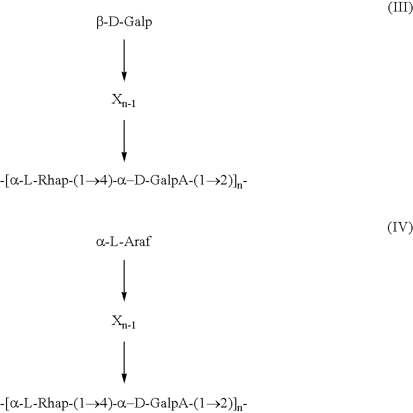
In the above representations, n is an integer greater than 1, Xₙ₋₁ represents a short side-chain of neutral sugar residues, X can be any of several sugars found in pectin side chains, including but not limited to β-Apif, β-Rhap, α-Fucp, β-GlcpA, α-GalpA, β-GalpA, β-DhapA, Kdop, β-Acef, α-Galp, and α-Araf.

It will be understood that natural pectin does not possess a strictly regular repeating structure, and that additional random variations are likely to be introduced by partial hydrolysis of the pectin, so that the identity of Ym and the values of n and o may vary from one iteration to the next of the p repeating units represented by formula II above.

An exemplary polymer of this type is modified pectin, preferably water soluble pH modified citrus pectin. Suitable polymers of this type are disclosed in, for example U.S. Pat. Nos. 5,834,442, 5,895,784, 6,274,566 and 6,500,807, and PCT Publication WO 03/000,118.

Pectin is a complex carbohydrate having a highly branched structure comprised of a polygalacturonic backbone with numerous branching side chains dependent therefrom. The branching creates regions which are characterized as being “smooth” and “hairy.” It has been found that pectin can be modified by various chemical, enzymatic or physical treatments to break the molecule into smaller portions having a more linearized, partially demethoxylated, polygalacturonic backbone with pendent side chains of rhamnose residues with terminal galactose residues, having decreased branching. The resulting partially depolymerized pectin is known in the art as modified pectin, and its efficacy in treating cancer has been established.

U.S. Pat. No. 5,895,784, the disclosure of which is incorporated herein by reference, describes modified pectin materials, techniques for their preparation, and use of the material as a treatment for various cancers. The material of the '784 patent is described as being prepared by a pH-based modification procedure in which the pectin is put into solution and exposed to a series of programmed changes in pH which results in the breakdown of the molecule to yield therapeutically effective modified pectin. The material in the '784 patent is most preferably prepared from citrus pectin; although, it is to be understood that modified pectins may be prepared from pectin from other sources, such as apple pectin. Also, modification may be done by enzymatic treatment of the pectin, or by physical processes such as heating. Further disclosure of modified pectins and techniques for their preparation and use are also found in U.S. Pat. No. 5,834,442, U.S. patent application Ser. No. 08/024,487, and U.S. application Ser. No. 11/093,268, the disclosures of which are incorporated herein by reference. Certain modified pectins of this type generally have molecular weights in the range of less than 100 kDa. A group of such materials has an average molecular weight of less than 3 kDa. Another group has an average molecular weight in the range of 1-15 kDa, with a specific group of materials having a molecular weight of about 10 kDa. In one embodiment, modified pectin has the structure of a pectic acid polymer with some of the pectic side chains still present. In preferred embodiments, the modified pectin is a copolymer of homogalacturonic acid and rhamnogalacturonan in which some of the galactose- and arabinose-containing side chains are still attached. More preferred embodiment of the present invention is a modified pectin composition that comprises or consists essentially of a homogalacturonan backbone with small amounts of rhamnogalacturonan interspersed therein, with neutral sugar side chains, and has a low degree of neutral sugar branching dependent from the backbone. In certain embodiments, the modified pectin is partially depolymerized, so as to have a disrupted homogalacturonan backbone. The modified pectin may have a molecular weight of 1 to 500 kilodaltons (kDa), preferably 10 to 250 kDa, more preferably 50-200 kDa, more preferably 70-200 kDa, even more preferably 70-150 kDa, and most preferably 80-100 kDa as measured by Gel Permeation Chromatography (GPC) with Multi Angle Laser Light Scattering (MALLS) detection.

Degree of esterification is another characteristic of modified pectins. Naturally occurring pectins are methoxylated so that the methoxyl groups account for up to 10% of the total mass of the pectin. Degree of methoxylation is a variable that can affect the biological and pharmacological activities of modified pectin. Modified pectins are demethoxylated to various degrees and contain reduced amounts of methoxyl groups compared to naturally occurring pectins.

Saccharide content is another characteristic of modified pectins. In certain embodiments, the modified pectin is composed entirely of a single type of saccharide subunit. In other embodiments, the modified pectin comprises at least two, preferably at least three, and most preferably at least four types of saccharide subunits. For example, the modified pectin may be composed entirely of galacturonic acid subunits. Alternatively, the modified pectin may comprise a combination of galacturonic acid and rhamnose subunits. In yet another example, the modified pectin may comprise a combination of galacturonic acid, rhamnose, and galactose subunits. In yet another example, the modified pectin may comprise a combination of galacturonic acid, rhamnose, and arabinose subunits. In still yet another example, the modified pectin may comprise a combination of galacturonic acid, rhamnose, galactose, and arabinose subunits. In some embodiments, the galacturonic acid content of modified pectin is greater than 50%, preferably greater than 60% and most preferably greater than 80%. In some embodiments, the rhamnose content is less than 25%, preferably less than 15% and most preferably less than 10%; the galactose content is less than 50%, preferably less than 40% and most preferably less than 30%; and the arabinose content is less than 15%, preferably less than 10% and most preferably less than 5%. In certain embodiments, the modified pectin may contain other uronic acids, xylose, ribose, lyxose, glucose, allose, altrose, idose, talose, gluose, mannose, fructose, psicose, sorbose or talalose in addition to the saccharide units mentioned above.

Modified pectin suitable for use in the subject methods may also have any of a variety of linkages or a combination thereof. By linkages it is meant the sites at which the individual sugars in pectin are attached to one another. In some embodiments, the modified pectin comprises only a single type of linkage. In certain preferred embodiments, the modified pectin comprises at least two types of linkages, and most preferably at least 3 types of linkages. For example, the modified pectin may comprise only alpha-1,4-linked galacturonic acid subunits. Alternatively, the modified pectin may comprise alpha-1,4-linked galacturonic acid subunits and alpha-1,2-rhamnose subunits. In another example, the modified pectin may be composed of alpha-1,4-linked galacturonic acid subunits and alpha-1,2-rhamnose subunits linked through the 4 position to arabinose subunits. In another example, the modified pectin may comprise alpha-1,4-linked galacturonic acid subunits and alpha-1,2-rhamnose subunits linked through the 4 position to arabinose subunits with additional 3-linked arabinose subunits. In another example, the modified pectin may comprise alpha-1,4-linked galacturonic acid subunits and alpha-1,2-rhamnose subunits linked through the 4 position to arabinose subunits with additional S-linked arabinose units. In another example, the modified pectin may comprise alpha-1,4-linked galacturonic acid subunits and alpha-1,2-rhamnose subunits linked through the 4 position to arabinose subunits with additional 3-linked and 5-linked arabinose subunits. In another example, the modified pectin may comprise alpha-1,4-linked galacturonic acid subunits and alpha-1,2-rhamnose subunits linked through the 4 position to arabinose subunits with additional 3-linked and 5-linked arabinose subunits with 3,5-linked arabinose branch points. In another example, the modified pectin may comprise alpha-1,4-linked galacturonic acid subunits and alpha-1,2-rhamnose subunits linked through the 4 position to galactose subunits. In another example, the modified pectin may comprise alpha-1,4-linked galacturonic acid subunits and alpha-1,2-rhamnose subunits linked through the 4 position to galactose subunits with additional 3-linked galactose subunits. In another example, the modified pectin may comprise alpha-1,4-linked galacturonic acid subunits and alpha-1,2-rhamnose subunits linked through the 4 position to galactose subunits with additional 4-linked galactose subunits. In another example, the modified pectin may comprise alpha-1,4-linked galacturonic acid subunits and alpha-1,2-rhamnose subunits linked through the 4 position to galactose subunits with additional 3-linked galactose subunits with 3,6-linked branch points. In another example, the modified pectin may comprise alpha-1,4-linked galacturonic acid subunits and alpha-1,2-rhamnose subunits linked through the 4 position to galactose subunits with additional 4-linked galactose subunits with 4,6-linked branch points. In certain embodiments, the side chains of the modified pectin may comprise uronic acids, galacturonic acid, glucuronic acid, rhamnose, xylose, ribose, lyxose, glucose, allose, altrose, idose, talose, gluose, mannose, fructose, psicose, sorbose or talalose in addition to the saccharide units described above.

In certain embodiments, the modified pectin preparation is a substantially ethanol-free product suitable for parenteral administration. By substantially free of ethanol, it is meant that the compositions of the invention contain less than 5% ethanol by weight. In preferred embodiments the compositions contain less than 2%, and more preferably less than 0.5% ethanol by weight. In certain embodiments, the compositions further comprise one or more pharmaceutically acceptable excipients. Such compositions include aqueous solutions of the modified pectin of the invention. In certain embodiments of such aqueous solutions, the pectin modification occurs at a concentration of at least 7 mg/mL, and preferably at least 10 or even 15 or more mg/ml. Any of such compositions are also substantially free of organic solvents other than ethanol.

Yet another class of compound useful to carry out the methods of present invention is galactomannan. Galactomannan is a polysaccharide comprising mannose backbone with galactose pendent therefrom. Galactomannan is found in nature and can be isolated from plant materials as well as from yeasts, having molecular weight in the range of 20-600 kDa, 90-415 kDa or 40-200 kDa depending on the source. In specific examples, the galactomannan may have an average molecular weight of 50, 83, or 215 kDa. In a preferred embodiment, the galactomannan may be a β-1,4 D-galactomannan. Moreover, the galactomannan may include a ratio of 2.0-3.0 mannose to 0.5-1.5 galactose. The ratio of mannose to galactose may be about 1.13 mannose to 1 galactose, 1.7 mannose to 1 galactose, 2.6 mannose to 1.5 galactose or 2.2 mannose to 1 galactose.

### B. BENZODIAZEPINE RECEPTOR LIGANDS

Ligands of benzodiazepine receptors of mitochondria have recently been found to induce apoptosis by affecting the permeability transition pore (“PT pore”) of the mitochondria. See, for example, U.S. Pat. No. 6,319,931, the disclosure of which is incorporated by reference in its entirety.

In a preferred embodiment, the ligand of PBR is selected from 1-(2-chlorophenyl)-N-methyl-N-(1-methylpropyl)-3-isoquino-linecarboxamide (PK11195), 7-chloro-5-(4-chlorophenyl)-1,3-dihydro-1-methyl-2-H-1,4-benzodiazepin-2 (4-chlorodiazepam or Ro5-4864), porphyrins, alpidem, N-(2,5-dimethoxybenzyl)-N-5-fluoro-2-phenoxyphenyl)acetamide (DAA1106), 2-aryl-3-indoleacetamide (FGIN-1), diazepam (Valium®), flumazenil (Romazicon®) or lonidamine. The ligand of PBR may also be protoporphyrin IX, mesoporphyrin IX, deuteroporphyrin IX, or other dicarboxylic acid porphyrins.

More preferably, the ligand of peripheral benzodiazepine receptor is PK11195.

### C. CHEMOTHERAPEUTIC AGENTS

A preferred chemotherapeutic agent to be administered in conjunction with the PBR ligand and the carbohydrate described herein is a topoisomerase inhibitor. A topoisomerase inhibitor may be adriamycin, amsacrine, camptothecin, daunorubicin, dactinomycin, doxorubicin, eniposide, epirubicin, etoposide, idarubicin, mitoxantrone, teniposide, or topotecan. Preferably, the topoisomerase inhibitor is etoposide.

### D. ADMINISTRATION

A composition of the present invention may be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams. Preferably, the route of administration is parenteral, more preferably intravenous.

In general, an embodiment of the invention is to administer a suitable daily dose of a therapeutic composition that will be the lowest effective dose to produce a therapeutic effect, for example, mitigating symptom. The therapeutic composition comprises the carbohydrate polymer and a ligand of PBR. Optionally, a chemotherapeutic agent such as a topoisomerase inhibitor is administered conjointly with the carbohydrate polymer and a ligand of PBR. The dosage of the therapeutic composition of the present invention is set at such amount that the composition does not induce psychoactive effects via its effect on the benzodiazepine receptor of the central nervous system. The carbohydrate polymer is administered at a dosage that improves the therapeutic index of a ligand of the PBR by at least two times, and more preferably ten times, and most preferably more than ten times compared to when the ligand is administered without the carbohydrate polymer. In general, the effective dosage of the carbohydrate polymer in the present invention is about 50 to about 400 micrograms of the compound per kilogram of the subject per day.

However, it is understood by one skilled in the art that the dose of the composition to practice the invention will vary depending on the subject and upon the particular route of administration used. It is routine in the art to adjust the dosage to suit the individual subjects. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point.

The therapeutic treatment may be administered hourly, daily, weekly, monthly, yearly (e.g., in a time release form) or as a one-time delivery. The delivery may be continuous delivery for a period of time, e.g., intravenous delivery. In one embodiment of the methods described herein, the therapeutic composition is administered at least once per day. In one embodiment, the therapeutic composition is administered daily. In one embodiment, the therapeutic composition is administered every other day. In one embodiment, the therapeutic composition is administered every 6 to 8 days. In one embodiment, the therapeutic composition is administered weekly.

The carbohydrate polymer described herein may be administered before, after, or at the same time with a ligand of PBR and/or a chemotherapeutic agent. In some instances, administration of the therapeutic carbohydrate material is commenced at least several days prior to the administration of the PBR ligand and/or a chemotherapeutic agent, while in other instances, administration is begun either immediately before or at the time of the administration of the PBR ligand and/or chemotherapeutic agent. In some instances, the carbohydrate material may be advantageously administered both before, during and after the therapy.

In one embodiment of the methods described herein, the route of administration can be oral, intraperitoneal, transdermal, subcutaneous, by vascular injection into the tumor, by intravenous or intramuscular injection, by inhalation, topical, intralesional, infusion; liposome-mediated delivery; intrathecal, gingival pocket, rectal, intrabronchial, nasal, transmucosal, intestinal, ocular or otic delivery, or any other methods known in the art as one skilled in the art may easily perceive. In other embodiments of the invention, the compositions incorporate particulate forms protective coatings, hydrolase inhibitors or permeation enhancers for various routes of administration, including parenteral, pulmonary, nasal and oral.

An embodiment of the method of present invention is to administer the carbohydrate polymer describes herein in a sustained release form. Such method comprises implanting a sustained-release capsule or a coated implantable medical device so that a therapeutically effective dose of the carbohydrate polymer is continuously delivered to a subject of such a method. The carbohydrate polymer may be delivered via a capsule which allows sustained-release of the agent or the peptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines).

### E. EXEMPLARY TARGETS OF THE TREATMENT

The method of present invention is effective in treatment of various types of cancers, including but not limited to: pancreatic cancer, renal cell cancer, Kaposi's sarcoma, chronic leukemia (preferably chronic myelogenous leukemia), chronic lymphocytic leukemia, breast cancer, sarcoma, ovarian carcinoma, rectal cancer, throat cancer, melanoma, colon cancer, bladder cancer, lymphoma, mesothelioma, mastocytoma, lung cancer, liver cancer, mammary adenocarcinoma, pharyngeal squamous cell carcinoma, gastrointestinal cancer, stomach cancer, myeloma, prostate cancer, B-cell malignancies or metastatic cancers.

The present invention is also effective against other diseases related to unwanted cell proliferation. Such hyperproliferative diseases include but are not limited to: psoriasis, rheumatoid arthritis, lamellar ichthyosis, epidermolytic hyperkeratosis, restenosis, endometriosis, proliferative retinopathy, lung fibrosis, desmoids or abnormal wound healing.

### F. EXAMPLES

The effect of GCS-100 on inducing apoptosis of cells was tested by alamarBlue (Biosource) assay using a mouse melanoma cell line B16F10. AlamarBlue is a mitochondrial enzyme indicator that can be used to measure the inhibition of proliferation of the B 16F 10 cells.

B16F10 cells were seeded into the wells of 96-well plates in growth media. After cells attached to the plates, 0, 5, 50, or 150 μl/ml of sterile 4-chlorodiazepam and 100 μl/ml GCS-100 were applied to the cells. The cells were incubated to allow viable cells to proliferate, and the media was replaced with fresh media supplemented with 10% alamarBlue. The cells were further incubated, then the state of oxidation of alamarBlue was determined by spectrophotometry and adjusted for blanks. The more reduced alamarBlue was, the higher the proportion of the cells that underwent apoptosis.

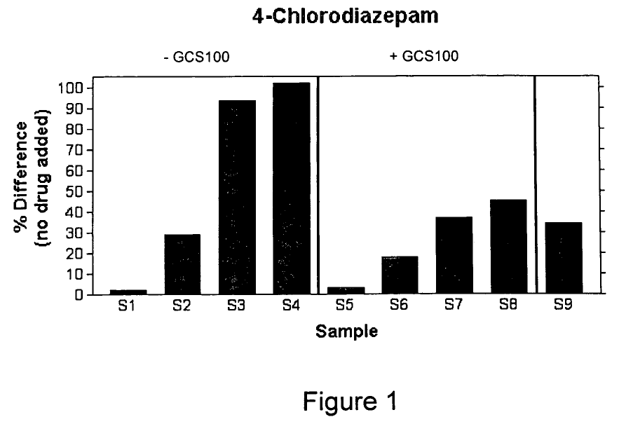

As seen in FIG. 1, concomitant administration of 4-chlorodiazepam and GCS-100 produce a synergistic effect, resulting in a larger proportion of cells manifesting apoptosis (sample S5-S8) compared to when 4-chlorodiazepam alone (sample S1-S4) was added to the cells. The concentration of 4-chlorodiazepam was 5 μg/ml for samples S2 and S6; 50 μl/ml for samples S3 and S7; and 150 μl/ml for samples S4 and S8. Samples S1 and S5 contained no 4-chlorodiazepam.

The foregoing discussion and description is illustrative of specific embodiments, but is not meant to be a limitation upon the practice thereof. It is the following claims, including all equivalents, which define the scope of the invention.

We claim:

1. A method for treating cancer and unwanted proliferation of cells in a patient, said therapeutic treatment comprising the conjoint administration to said patient of a therapeutically effective amount of ligand of a peripheral benzodiazepine receptor and a carbohydrate having a partially demethoxylated polygalacturonan backbone.

2. The method of claim 1, wherein said backbone comprises homogalacturonan.

3. The method of claim 1, wherein said backbone comprises a partially demethoxylated polygalacturonic acid which is interrupted with rhamnose residues.

4. The method of claim 3, wherein said carbohydrate further comprises neutral sugar side chains having a low degree of branching pendent from the backbone.

5. The method of any of claims 1 to 4, wherein said ligand is selected from:
1-(2-chlorophenyl)-N-methyl-N-(1-methylpropyl)-3-isoquino-linecarboxamide (PK11195), 7-chloro-5-(4-chlorophenyl)-1,3-dihydro-1-methyl-2-H-1,4-benzodiazepin-2 (4-chlorodiazepam or Ro5-4864), porphyrins, alpidem, N-(2,5-dimethoxybenzyl)-N-5-fluoro-2-phenoxyphenyl)acetamide (DAA1106), 2-aryl-3-indoleacetamide (FGIN-1), diazepam (Valium), flumazenil (Romazicon) or lonidamine.

6. The method of any of claims 1 to 4, further comprising a chemotherapeutic agent.

7. The method of claim 6, wherein said chemotherapeutic is a topoisomerase inhibitor.

8. The method of claim 7, wherein said topoisomerase inhibitor is selected from the group consisting of adriamycin, amsacrine, camptothecin, daunorubicin, dactinomycin, doxorubicin, eniposide, epirubicin, etoposide, idarubicin, mitoxantrone, teniposide, and topotecan.

9. The method of claim 8, wherein said inhibitor is etoposide.

10. The method of claim 1, used in the treatment of a proliferative disorder selected from the group consisting of a pancreatic cancer, renal cell cancer, Kaposi's sarcoma, chronic leukemia, chronic lymphocytic leukemia, breast cancer, sarcoma, ovarian carcinoma, rectal cancer, throat cancer, melanoma, colon cancer, bladder cancer, lymphoma, mesothelioma mastocytoma, lung cancer, liver cancers, mammary adenocarcinoma, pharyngeal squamous cell carcinoma, gastrointestinal cancer, stomach cancer, myeloma, prostate cancer, B-cell malignancies or metastatic cancers.

11. The method of claim 1, used to inhibit growth of a tumor cell selected from the group consisting of a pancreatic tumor cell, a lung tumor cell, a prostate tumor cell, a breast tumor cell, a colon tumor cell, a liver tumor cell, a brain tumor cell, a kidney tumor cell, a skin tumor cell and an ovarian tumor cell.

12. The method of claim 1, used to inhibit growth of a tumor cell selected from the group consisting of a squamous cell carcinoma, a non-squamous cell carcinoma, a glioblastoma, a sarcoma, an adenocarcinoma, a myeloma, a melanoma, a papilloma, a neuroblastoma and a leukemia cell.

13. The method of claim 1, wherein said carbohydrate is a partially depolymerized pectin.

14. The method of claim 13, wherein said partially depolymerized pectin comprises a pH modified pectin, an enzymatically modified pectin, or a thermally modified pectin.

15. The method of claim 13, wherein said partially depolymerized pectin comprises a modified citrus pectin.

16. The method of claim 13, wherein said partially depolymerized pectin has a molecular weight of 1 to 500 kilodaltons (kDa).

17. The method of claim 13 wherein said partially depolymerized pectin has a molecular weight of 10 to 250 kDa.

18. The method of claim 13 wherein said partially depolymerized pectin has a molecular weight of 50 to 200 kDa.

19. The method of claim 13 wherein said partially depolymerized pectin has a molecular weight of 70 to 150 kDa.

20. The method of claim 13 wherein said partially depolymerized pectin has a molecular weight of 80 to 100 kDa.

21. The method of claim 13, wherein said partially depolymerized pectin is administered by intravenous infusion.

22. The method of claim 13, wherein said partially depolymerized pectin is administered orally.

23. The method of claim 13, wherein said partially depolymerized pectin is administered by intramuscular or intraperitoneal injection or infusion.

24. The method of claim 13, wherein said partially depolymerized pectin is administered by inhalation.

25. The method of claim 13, wherein said partially depolymerized pectin is administered by topically.

26. The method of claim 13, wherein said partially depolymerized pectin is administered by subcutaneous injection.

27. A kit comprising (i) a ligand of peripheral benzodiazepine receptor, (ii) a therapeutically effective amount of a carbohydrate having a galacturonan backbone which may be disrupted by rhamnose and low branched neutral sugars dependent from said backbone; and (iii) instructions and/or a label for conjoint administration of the ligand and the carbohydrate.
